(19) United States
(12) Patent Application Publication
(10) Pub. No.: US 2004/0204383 A1
(43) Pub. Date: Oct. 14, 2004

(54) Lysophosphatidic acid analogs and inhibition of neointima formation

(76) Inventors: Gabor Tigyi, Memphis, TN (US); Daniel L. Baker, Memphis, TN (US); Chunxiang Zhang, Memphis, TN (US)

Correspondence Address: Benjamin Aaron Adler, ADLER & ASSOCIATES, 8011 Candle Lane, Houston, TX, 77071, US

(21) Appl. No.: 10/821,739

(22) Filed: Apr. 9, 2004

Related U.S. Application Data
(60) Provisional application No. 60/462,274, filed on Apr. 11, 2003.

(51) Int. Cl. A61K 31/739

(52) U.S. Cl. 514/54

(57) ABSTRACT

The phospholipid growth factor lysophosphatidic acids (LPAs) containing unsaturated fatty acids (18:1, 18:2 and 20:4) and fatty alcohols containing hydrocarbon chains with more than 4 carbons were capable of inducing a rapid formation of neointima, an initial step in the development of atherosclerotic plaque. LPAs with saturated fatty acids did not induce neointima formation. A Peroxisome Proliferator-Activated Receptors gamma (PPARγ)-specific agonist Rosiglitasone also induced a profound formation of neointima. GW9662, a selective and irreversible antagonist of PPARγ, abolished LPA- and Rosiglitazone-induced neointima formation, indicating that LPA-induced neointima formation requires the activation of PPARγ. These data suggest that LPA analogs that bind to but do not activate downstream signaling of PPARγ or antagonists of PPARγ that inhibit PPARγ signaling would be useful in the prevention and/or treatment of neointima formation and atherosclerosis.

### CROSS-REFERENCE TO RELATED APPLICATION

This non-provisional patent application claims benefit of provisional patent application U.S. Serial No. 60/462,274, filed Apr. 11, 2003, now abandoned.

### FEDERAL FUNDING LEGEND

This invention was produced in part using funds obtained through grants HL061469, CA92160, and HL070231 from the National Institutes of Health. Consequently, the federal government has certain rights in this invention.

### BACKGROUND OF THE INVENTION

1. Field of the Invention

The present invention relates generally to the field of vascular biology. More specifically, the present invention relates to the role of lysophosphatidic acid and PPARγ (peroxisome proliferator-activated receptor gamma) in neointima formation and atherogenesis.

2. Description of the Related Art

Atherosclerosis is the leading cause of death in the United States and is responsible for more than half of all mortality in developed countries. Neointima formation is the initial step in the development of the atherosclerotic plaque. Several cellular mechanisms are involved in neointima formation including platelet activation and thrombus formation, endothelial cell activation and injury, infiltration by inflammatory cells, and activation, migration, phenotypic modulation, and proliferation of vascular smooth muscle cells. Neointima proliferation ultimately leads to neointimal-plaque formation, lipid accumulation and calcification. Rupture of the inflamed atheromatous-plaque can trigger acute thrombembolic events which are direct causes of heart attack and stroke.

Lysophosphatidic Acid in Vascular Biology

Lysophosphatidic acid (LPA) is a growth factor-like phospholipid mediator that acts through the G protein-coupled plasma membrane receptors LPA₁, LPA₂, and LPA₃ encoded by the Endothelial Differentiation Gene family. The cellular responses elicited by lysophosphatidic acid include proliferation, migration, de-differentiation and anti-apoptotic effects—responses that are all involved in neointima formation. Production of lysophosphatidic acid is tied to platelet activation and involves two enzymatic steps. First, activated platelets release phospholipase A₁ and A₂ enzymes, which hydrolyze plasma and membrane phospholipids and generate lysophospholipids. Subsequently, the de novo generated lysophospholipids become substrates for lysophospholipase D that produces lysophosphatidic acid through cleavage of phosphate head groups. Lysophosphatidic acid generated by this metabolic pathway is enriched in the 18:2 and 20:4 polyunsaturated fatty acyl forms.

Platelet activation is the trigger for lysophosphatidic acid formation in blood. The concentration of lysophosphatidic acid in plasma is in the nanomolar range, whereas in serum as a result of platelet activation it increases 100 fold to as high as 10 μM. In addition to the platelet-linked mechanism, minimally oxidized LDL (mox-LDL) also contains lysophosphatidic acid-like activity. Lipid-rich atheromatous plaques, which accumulate oxidized lipids including mox-LDL, contain several acyl- and alkyl species of lysophosphatidic acid. Lysophosphatidic acid is a potent activator of platelet aggregation. Lysophosphatidic acid formed during mild oxidation of LDL is one of the key mediators responsible for platelet activation induced by minimally oxidized LDL. In the event of plaque rupture, circulating platelets come into contact with this highly thrombogenic material. Indeed, oxidatively modified LDL and lipid extracts of human atherosclerotic plaques have been shown to stimulate platelets.

Platelet activation elicited by lysophosphatidic acid, minimally oxidized LDL and plaque lipids can be blocked by diacylglycerol pyrophosphate (DGPP), an LPA_{1&3} selective antagonist. Oxidized LDL is also present in circulating blood and may be responsible for enhanced aggregation of platelets and the prothrombotic state often observed in patients with cardiovascular disease. Lysophosphatidic acid and minimally oxidized LDL are capable of activating endothelial cells, macrophages and modulating endothelial/leukocyte interactions. DGPP and another lysophosphatidic acid receptor antagonist, N-palmitoyl-serine phosphoric acid (NPSerPA), inhibit the effects of lysophosphatidic acid on platelets and endothelial cells.

Lysophosphatidic acid has been identified as a potent mitogen and motility factor for vascular smooth muscle cells (VSMC). It has been reported that lysophosphatidic acid induced vascular smooth muscle cell dedifferentiation in vitro. Only lysophosphatidic acid containing unsaturated fatty acids (for example, but not limited to, 18:1, 18:2 and 20:4) and fatty alcohols with hydrocarbon chains in excess of 4 and not saturated fatty acids (16:0, 18:0) are capable of inducing vascular smooth muscle cell dedifferentiation. This selectivity for unsaturated lysophosphatidic acid species does not match the selectivity of any known lysophosphatidic acid plasma membrane receptors, as those receptors are activated by both saturated and unsaturated lysophosphatidic acid analogs. Therefore, it is hypothesized that a novel lysophosphatidic acid receptor must mediate the dedifferentiation effect.

An Intracellular Receptor for Lysophosphatidic Acid

It was reported recently that peroxisome proliferator-activated receptor-γ (PPARγ) was an intracellular receptor for lysophosphatidic acid. Peroxisome proliferator-activated receptors (PPARs) are transcription factors of the nuclear receptor superfamily. The peroxisome proliferator-activated receptor family consist of three isoforms: PPARα, PPARδ, and PPARγ. Binding of peroxisome proliferator-activated receptor ligands leads to activation and heterodimerization with retinoic acid X receptor (RXR), the receptor for 9-cis-retinoic acid. PPAR/RXR heterodimers bind to specific peroxisome proliferator response elements (PPRE) located upstream of responsive genes. Although these three isoforms belong to the same superfamily, their biological actions are distinct.

Peroxisome proliferator-activated receptors can be activated by a vast number of compounds including synthetic drugs such as Rosiglitazone and Troglitazone, oxidized phospholipids, fatty acids, eicosanoids, and oxidized LDL. The actions of peroxisome proliferator-activated receptors were originally thought to be limited to the control of lipid metabolism and homeostasis. Recent studies, however, have shown that peroxisome proliferator-activated receptor activation regulates inflammatory responses, cellular proliferation, differentiation, and apoptosis. Interestingly, unsaturated 18:1 lysophosphatidic acid but not saturated 18:0 binds to PPAR-γ and transactivates peroxisome proliferator responsive elements. PPARγ is expressed in monocytes/macrophages, vascular smooth muscle cells, endothelial cells, and is highly expressed in atherosclerotic lesions and hypertensive vascular wall. The role of PPARγ in vascular diseases remains unclear.

A Novel Hypothesis for the Role of Lysophosphatidic Acid in Atherogenesis

It has been proposed that local activation of platelets at hemodynamically compromised sites leads to platelet activation and release of platelet-borne mediators. Activated platelets release enzymatic activities involved in lysophosphatidic acid biosynthesis. Locally produced lysophosphatidic acid activates the expression of adhesion molecules including E-selectin and VCAM, which in turn leads to increased platelet adhesion. Because lysophosphatidic acid also activates platelets, this positive feedback mechanism likely recruits more platelets, as well as stimulates the generation of lysophosphatidic acid enriched in unsaturated fatty acid species.

Although many lipids can activate PPARγ, lysophosphatidic acid stands out as its concentration can rise to 10 μM in serum and concentrations near the vessel wall are likely to be even higher. Locally generated lysophosphatidic acid, due to its lipophilic character, is able to cross the plasma membrane and combine with PPARγ in the cytoplasm. The lysophosphatidic acid-PPARγ complex translocates to the nucleus and transactivates genes with peroxisome proliferator response elements in their promoters. Of these genes the scavenger receptor gene, CD36, is perhaps the most interesting, as it is involved in lipid import into the cell including the uptake of minimally oxidized LDL. Increased expression of CD36 at the site of atheromatous plaques is well documented. Lysophosphatidic acid has been found to upregulate CD36 transcription. Increased CD36 expression which leads to increased minimally oxidized LDL uptake into the cell might provide a sustained source of lysophosphatidic acid and other activating lipids of PPARγ.

The above data and hypothesis suggest lysophosphatidic acid plays an important role in the pathogenesis of atherosclerosis. The prior art, however, is deficient in delineating the role of lysophosphatidic acid in atherosclerosis. The present invention fulfills this outstanding need in the art by testing various key aspects of the above mentioned hypothesis and assigns a pivotal role to lysophosphatidic acid in atherogenesis.

### SUMMARY OF THE INVENTION

Experiments disclosed herein examined whether lysophosphatidic acid (LPA)-induced activation of PPARγ in mice and rats could account for neointima formation. The external carotid arteries of male Sprague-Dawley rats or C57B6 mice were canulated and the original common and internal carotid arteries were clipped. LPA or solvent was infused into the vessel over a period of 30-60 min. The circulation was restored and the animals were allowed to survive for 7 to 56 days post surgery before histological evaluation of the treated vessels was done.

The results showed that low micromolar concentrations (1-10 μM) of lysophosphatidic acids (LPAs) with unsaturated fatty acids (18:1, 18:2 and 20:4) were capable of inducing a rapid formation of neointima. The EDG receptor ligands 18:0 LPA, 16:0 lysophosphatidic acid, 18:1 cyclic-phosphatidic acid and the LPA₃ receptor-selective ligand [1-Fluoro-3 (S)-hydroxyl-4-(oleoyloxy)butyl]phosphonate were not active. In contrast, fluorinated analogs of lysophosphatidic acid, which do not activate the EDG receptors in low micromolar concentrations, and the PPARγ receptor-specific agonist Rosiglitasone (3-10 μM) induced a profound formation of neointima. Polypeptide growth factors PDGF (10 ng/ml), EGF (100 ng/ml), and VEGF (10 ng/ml) were ineffective in inducing neointima formation. GW9662, a selective and irreversible antagonist of PPARγ, abolishes LPA- and Rosiglitazone-induced neointima formation, indicating that lysophosphatidic acid-induced neointima formation requires the activation of PPARγ. Taken together, these data suggest that lysophosphatidic acid analogs that bind to but do not activate downstream signaling of PPARγ or antagonists of PPARγ that inhibit PPARγ signaling would be useful in the prevention and/or treatment of neointima formation and atherosclerosis.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows the structural formulas of lipid mediators used in the following experiments.

FIGS. 2A-C show minimally oxidized low density lipoprotein (moxLDL) treatment induces neointima formation in rat carotid arteries. The figures show representative views of Masson's trichrome-stained, paraffin-embedded sections from animals treated with native low density lipoprotein (nLDL) (FIG. 2A) or moxLDL (FIG. 2B) 2 wk after a 1 h treatment with 5 mg LDL protein/ml. Bar, 500 μm. Intima to media ratios were quantified in FIG. 2C (n=5).

FIGS. 3A-B show five most abundant acyl-lysophosphatidic acid (LPA) (FIG. 3A) and alkyl-GP (FIG. 3B) species quantified in native low density lipoprotein (nLDL) and minimally oxidized low density lipoprotein (moxLDL) using stable isotope dilution electrospray ionization mass spectrometry. The lack of difference in the total acyl-lysophosphatidic acid content between native low density lipoprotein and minimally oxidized low density lipoprotein is in sharp contrast to the six-fold increase in alkyl-GP levels in minimally oxidized low density lipoprotein (n=4). In the batch of native low density lipoprotein used in the experiments shown in FIGS. 2 and 3, alkyl-GP concentration was 0.1 μM, and the total concentration of unsaturated lysophosphatidic acid plus alkyl-GP was 0.5 μM, whereas in minimally oxidized low density lipoprotein these concentrations were 0.7 and 0.9 μM, respectively.

FIG. 3C shows structure-activity relationship of neointimal lesion induction for various acyl-lysophosphatidic acid (10 μM) and alkyl-GP (AGP) species (10 μM). Only select LPA species elicit neointima as the effect was stereoselective with a preference for 1AGP over 3AGP. LPA 18:0 and cPA 18:1 did not elicit detectable neointima.

FIG. 4A shows exposure of rat carotid arteries for 1 h to 2.5 μM LPA 20:4, but not to lysophosphatidic acid, 20:0 elicited progressive growth of neointima that continued for up to 8 wk post-treatment. Groups of 5 animals each were used for quantitative morphometric analysis.

FIG. 4B shows dose-response curve for lysophosphatidic acid 18:1-elicited neointimal response. Mean (±SE) intima to media ratios were determined for groups of five animals 2 wk after treatment.

FIG. 5A shows RT-PCR of lysophosphatidic acid (LPA)-specific G protein-coupled plasma membrane receptors in the rat carotid tissue. LPA₁, LPA₄, and S1P1 were detected in RNA extracted from whole carotid tissue.

FIG. 5B shows the effects of polypeptide growth factors and non-lysophosphatidic acid G protein-coupled receptor ligands fluorinated lysophosphatidic acid analogs on neointima formation. Animals treated with 10 μM lysophosphatidic acid 18:1, XY4, and its regioisomer XY8 but not those treated with EGF (50 ng/ml), VEGF (10 ng/ml), PDGF-BB (10 ng/ml), or lysophosphatidic acid 18:0 (10 μM) showed neointima formation. Groups of five animals were treated with the compounds.

FIG. 5C shows RT-PCR analysis detected PPARα, PPARδ, and PPARγ transcripts in normal carotid tissue.

FIG. 6A shows pertussis toxin (PTX) and dioctylglycerol pyrophosphate (DGPP), inhibitors of lysophosphatidic acid G protein-coupled receptor signaling, partially attenuated neointima formation induced by lysophosphatidic acid 20:4, whereas the PPARγ-specific antagonist GW9662 completely abolished this effect.

FIG. 6B shows Rosi, 1-O-hexadecyl-2-azale-oly-phosphatidylcholine (AZ-PC), minimally oxidized LDL, and unsaturated acyl forms of lysophosphatidic acid all induced neointima formation that was completely abolished by GW9662. In contrast, stearoyl-oxovaleryl phosphatidylcholine (SOV-PC), a PPARγ-selective agonist, was ineffective in stimulating the development of neointima after 2 weeks.

FIG. 6C shows in vitro assay using CV1 cells transfected with PPARγ and a PPRE-Acox-Rluc reporter gene showed an identical structure-activity relationship when exposed to different lysophosphatidic acid species as found for the same set of ligands in the neointima assay in vivo (see FIG. 3C).

FIG. 6D shows the PPARγ antagonist GW9662 (10 μM) abolished, whereas PTX (100 ng/ml, 2 h) and DGPP (10 μM, 2 h) pretreatment and coapplication with lysophosphatidic acid partially inhibited lysophosphatidic acid 20:4-induced PPRE-Acox-Rluc reporter gene expression in vitro. Vehicle contained 1% DMSO and 10 μM BSA in PBS, Rosi (10 μM), lysophosphatidic acid 20:0 (10 μM), or lysophosphatidic acid 20:0 (10 μM) were applied for 20 h. Luciferase and β-galactosidase activities (mean±SEM) were measured in the cell lysate (n=4).

FIGS. 6E-F show dose-response relationship of LPA 20:4- and Rosi-induced activation of PPRE-Acox-Rluc reporter gene expression in vitro. *P>0.05 and **P>0.01, significant differences over vehicle control.

FIGS. 7A-D show immunohistological staining for PPARγ in rat carotid arteries. Only a few nuclei show PPARγ immunoreactivity in a carotid artery 4 wk after treatment with 2.5 μM LPA 20:0 (FIG. 7A). In contrast, the multilayered neointima elicited by LPA 20:4 expresses high levels of PPARγ immunoreactivity (FIG. 7B). Activation of PPARγ within neointima in LPA 20:4-treated carotid arteries is indicated by the strong expression of CD36 in a distribution that overlaps that of PPARγ (FIG. 7D). Little immunoreactivity for CD36 was noted in LPA 20:0-treated animals (FIG. 7C). Anti-PPARγ and anti-CD36 were from Santa Cruz Biotechnology, Inc. Bars, 250 μm.

FIG. 7E shows stimulation of CD36(−273)-Rluc and CD36(−261)-Rluc reporter genes by Rosi, LPA, and AGP in CV-1 cells. Rosi and LPA 20:4 but not LPA 20:0 (all 10 μM) elicited significant stimulation of CD36(−273)-Rluc that contains a PPRE between bp −273 and −261. Neither compound caused stimulation of the CD36(−261)-Rluc. 1AGP showed higher stimulation of the Rluc reporter compared with 3AGP.

FIG. 7F shows plasma and serum factors inhibit Rosi- and lysophosphatidic acid 20:4-induced neointimal lesion formation in rat carotid arteries. Rosi (10 μM) and lysophosphatidic acid 20:4 (2.5 μM) elicited neointima formation 2 wk after treatment when delivered as BSA complexes. In contrast, when the compounds were delivered in rat plasma or serum no neointima formation was detected (n=5).

FIGS. 7G-H show albumin (0.04%-4%, FIG. 7G) or rat serum (1-20%, FIG. 7H) inhibited lysophosphatidic acid- and Rosi-induced activation of the PPRE-Acox-Rluc reporter gene in a concentration-dependent manner.

FIGS. 8A-I show PPARγ agonists elicit phenotypic modulation and dedifferentiation of VSMCs in vitro. VSMC cultures established in the presence of 2 ng/ml IGF-1 (FIG. 8A) were treated with 1 μM each of lysophosphatidic acid 20:0 (FIG. 8C), lysophosphatidic acid 20:4 (FIG. 8E), and Rosi (FIG. 8G) for 3 d. LPA 20:4 and Rosi treatments lead to a pronounced change in the morphology of VSMCs. Pretreatment of the cultures with 200 nM GW9662 for 30 min did not affect the spindle-like morphology of the IGF-1- (FIG. 8B) and LPA 20:0-treated cultures (FIG. 8D). In contrast, GW9662 reversed the flattened morphology into a spindle-like shape in cultures treated with LPA 20:4 (FIG. 8F) and Rosi (FIG. 8H). Calibration bar=100 μm. Expression of heavy caldesmon (hCAD) mRNA decreased significantly by day 5 in VSMCs treated with Rosi and lysophosphatidic acid (FIG. 8I, white bars) compared with the IGF-treated control cultures. This trend was reversed in cultures pretreated with 200 nM GW9662 (FIG. 8I, black bars) as the PPARγ antagonist caused a significant increase in the abundance of hCAD mRNA measured by quantitative RT-PCR (P>0.01, ANOVA).

FIG. 9 shows lead structures for PPARγ ligand screening (R=hydrocarbon chain).

### DETAILED DESCRIPTION OF THE INVENTION

Abbreviations used in the present invention: 1AGP, 1-octadecenyl-glycerophosphate; 3AGP, 3-O-octadecenyl-glycerophosphate; Acox, acyl-CoA oxidase; alkyl-GP, alkyl ether glycerophosphate; AZ-PC, 1-O-hexadecyl-2-azaleoyl-phosphatidylcholine; CCA, common carotid artery; cPA, 2,3-cyclic phosphatidic acid; DGPP, dioctylglycerol pyrophosphate; EGF, epidermal growth factor; GPCR, G protein-coupled receptor; hCAD, heavy caldesmon; IGF, insulin-like growth factor; LDL, low density lipoprotein; LPA, lysophosphatidic acid; moxLDL, minimally oxidized LDL; nLDL, native LDL; PAF, platelet-activating factor; PDGF, platelet-derived growth factor; PPAR, peroxisome proliferator-activated receptor; PPRE, PPAR response element; PTX, pertussis toxin; Rluc, renilla luciferase; Rosi, Rosiglitazone; S1P, sphingosine 1-phosphate; SOV-PC, stearoyl-oxovaleryl phosphatidylcholine; TZD, thiazolidinedione; VEGF, vascular epithelial growth factor; VSMC, vascular smooth muscle cell; XY4,1,1-difluorodeoxy-(2R)-palmitoyl-sn-glycero-3-phosphate; XY8,1-palmitoyl-(2R)-fluorodeoxy-sn-glycero-3-phosphate.

The focus of the present invention is the phospholipid growth factor lysophosphatidic acid (LPA). Data presented in this application show that lysophosphatidic acid elicits neointima formation through activation of Peroxisome Proliferator-Activated Receptor-γ (PPARγ). These results strongly indicate that lysophosphatidic acid acts as a mediator of atherogenesis via mechanisms involving PPARγ. These data also suggest that lysophosphatidic acid analogs that bind to but do not activate downstream signaling of PPARγ or antagonists of PPARγ that inhibit PPARγ signaling would be useful in the prevention and/or treatment of neointima formation and atherosclerosis. For example, the present invention demonstrated that GW9662, a selective and irreversible antagonist of PPARγ, abolishes neointima formation induced by lysophosphatidic acid or Rosiglitazone, a PPARγ receptor-specific agonist.

It will be appreciated by those skilled in the art that the present invention extends to prophylaxis as well as treatment of established diseases or symptoms. Moreover, it will be appreciated that the amount of antagonists of PPARγ or lysophosphatidic acid analogs required for use in treatment will vary with the nature of the condition being treated and the age and condition of the patient. The dosage will be determined ultimately at the discretion of the attendant physician or veterinarian. In general, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day.

While it is possible that antagonists of PPARγ or lysophosphatidic acid analogs may be therapeutically administered as raw chemical, it is preferable to present the active ingredient as a pharmaceutical formulation. The formulations according to the present invention may contain between 0.1-99% of active ingredient, conveniently from 30-95%. Accordingly, the present invention further provides for a pharmaceutical formulation comprising an antagonist of PPARγ or lysophosphatidic acid analog and a pharmaceutically acceptable salt or solvent, preferably together with one or more pharmaceutically acceptable carriers and, optionally, other therapeutic and/or prophylactic ingredients.

Formulations of the present invention include those especially formulated for oral, buccal, parenteral, transdermal, inhalation, intranasal, transmucosal, implant, or rectal administration. Oral administration, however, is preferred. For buccal administration, the formulation may take the form of tablets or lozenges formulated in conventional manner. Tablets and capsules for oral administration may contain conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. The tablets may be coated according to methods well known in the art.

Alternatively, the antagonists of PPARγ or lysophosphatidic acid analogs may be incorporated into oral liquid preparations such as aqueous or oily suspensions, solutions, emulsions, syrups or elixirs. Moreover, formulations containing these compounds may be presented as a dry product or power for constitution with water or other suitable vehicle before use. Such liquid preparations may contain conventional additives (e.g. suspending agents such as sorbitol syrup, methyl cellulose, glucose/sugar syrup, gelatin, hydroxyethylcellulose, carboxymethyl cellulose, aluminum stearate gel or hydrogenated edible fats), emulsifying agents (e.g. lecithin, sorbitan mono-oleate or acacia), non-aqueous vehicles (which may include edible oils such as almond oil, fractionated coconut oil, oily esters, propylene glycol or ethyl alcohol) and preservatives such as methyl or propyl p-hydroxybenzoates or sorbic acid. Such preparations may also be formulated as suppositories, e.g., containing conventional suppository bases such as cocoa butter or other glycerides. Additionally, formulations of the present invention may be formulated for parenteral administration by injection or continuous infusion. Formulations for injection may take such forms as suspensions, solutions, or emulsions in oily or aqueous vehicles, and may contain formulatory agents such as suspending, stabilising and/or dispersing agents.

The formulations comprising antagonists of PPARγ or lysophosphatidic acid analogs may also be formulated as a depot preparation. Such long acting formulations may be administered by implantation (for example, subcutaneously or intramuscularly) or by intramuscular injection. Accordingly, the compounds of the present invention may be formulated with suitable polymeric or hydrophobic materials (e.g. as an emulsion in an acceptable oil), ion exchange resins or as sparingly soluble derivatives such as a sparingly soluble salt.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Materials and Reagents

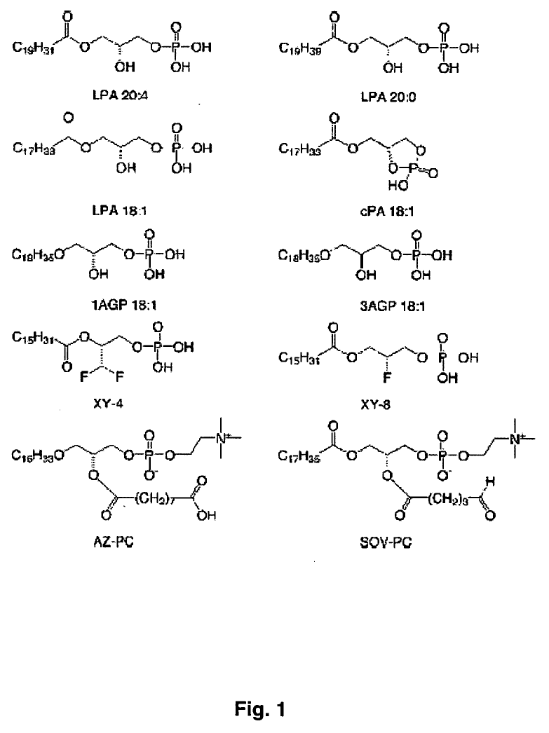

Lysophosphatidic acid (LPA) and sphingosine-1-phosphate (S1P) were from Avanti Polar Lipids. GW9662 was from Tocris Cookson Inc. Rosi was from ARC Inc. Stereoisomers of alkyl-glycerophosphate (alkyl-GP), LPA 18:2, 18:3, 20:0, 20:4, fluorinated lysophosphatidic acid analogs, including 1,1-difluorodeoxy-(2R)-palmitoyl-sn-glycero-3-phosphate (XY4), its regioisomer 1-palmitoyl-(2R)-fluorodeoxy-sn-glycero-3-phosphate (XY8; FIG. 1), and 1-O-hexadecyl-2-azale-oly-phosphatidylcholine (AZ-PC) (FIG. 1) were synthesized as described previously (Davies et al., 2001; Yokoyama et al., 2002; Xu and Prestwich, 2002) and provided by Echelon Biosciences Inc. Stearoyl-oxovaleryl phosphatidylcholine (SOV-PC) (FIG. 1) was from Dr. Judy Berliner (University of California, Los Angeles, Los Angeles, Calif.).

Native low density lipoprotein (nLDL) was purchased from Sigma-Aldrich, freed of EDTA by desalting on PD10 columns (Amersham Biosciences), and oxidized using Cu²⁺ as a catalyst. Protein concentrations were determined using the BCA protein assay kit (Pierce Chemical Co.). Final LDL concentrations were adjusted to 5 mg/ml before use. Plasma LDL concentration is 1.6 mg/ml but in patients can be as high as 8±6 mg/ml.

### EXAMPLE 2

Induction of Neointima Formation in Rat

Topical application of the test compounds was performed using the model developed and characterized recently by Yoshida et al. (2003). Briefly, the right carotid artery of anesthetized adult male Sprague-Dawley rats (250-300 g) was surgically exposed. The caudal origin of the common carotid artery was ligated using a vessel clip, followed by exposure and ligation of the internal carotid artery above the bifurcation. The external carotid artery was exposed, and a polyethylene catheter was inserted such that it never reached the common carotid artery, thereby avoiding mechanical injury to the vessel. The clip occluding the common carotid artery was temporarily released, and the vessel was rinsed with a retrograde injection of 500 μl physiological saline to remove residual blood. The vessel was again clipped, and 100 μl of treatment solution was injected. After 60 min of incubation, the cannula was withdrawn, the external carotid artery was ligated, and blood flow was restored. Animals were allowed to recover and were killed 7-56 d later by intracardiac perfusion of 10% buffered formaldehyde (pH 7.4). The common carotid artery from the jugular arch to the bifurcation was dissected, embedded in paraffin, and processed for histological analysis. Five μm-thick sections were cut and stained with hematoxylin and eosin or Masson's trichrome stain. Intima to media ratios were quantified using an image analysis system (Scion Image CMS-800).

### EXAMPLE 3

Effects of Oxidatively Modified Low Density Lipoprotein on Vascular Remodeling

Native low density lipoprotein (nLDL) is a transporter of phospholipids and cholesterol in blood. Oxidative modification of native low density lipoprotein by stresses such as cigarette smoke exposure results in the generation of novel lipid mediators. This process renders the resulting minimally oxidized LDL (moxLDL) highly atherogenic.

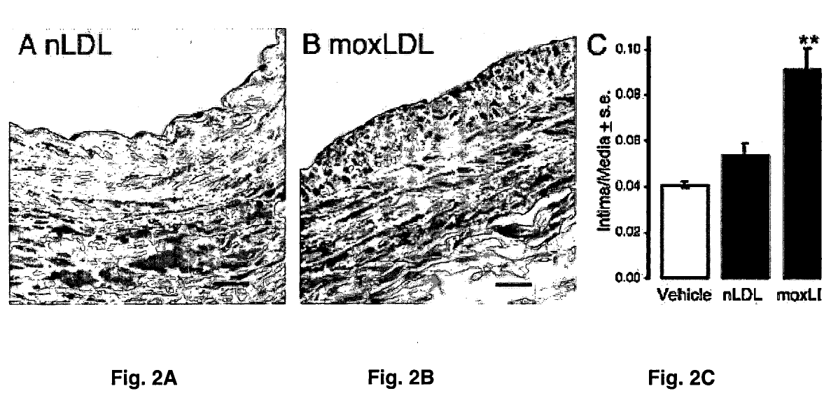

Common carotid arteries in rat were treated in situ as described above for 1 hour with native low density lipoprotein and minimally oxidized low density lipoprotein. Two weeks after treatment, carotid arteries were dissected en bloc and processed for histological evaluation. Minimally oxidized low density lipoprotein, but not native low density lipoprotein, elicited pronounced and significant neointima formation as illustrated in FIG. 2.

### EXAMPLE 4

Lysophosphatidic Acid Levels in Atherogenic Minimally Oxidized Low Density Lipoprotein

Minimal oxidization of low density lipoprotein generates lysophosphatidic acid-like biological activity. Lysophosphatidic acid elicits numerous effects on cells of the cardiovascular system including stimulation of platelet aggregation, activation of macrophages and endothelial cells, and the dedifferentiation and proliferation of vascular smooth muscle cell. Many of these lysophosphatidic acid-elicited cellular effects are implicated in the development of neointima lesions. Therefore, it is hypothesized that oxidative modification of LDL increases lysophosphatidic acid levels in atherogenic minimally oxidized LDL.

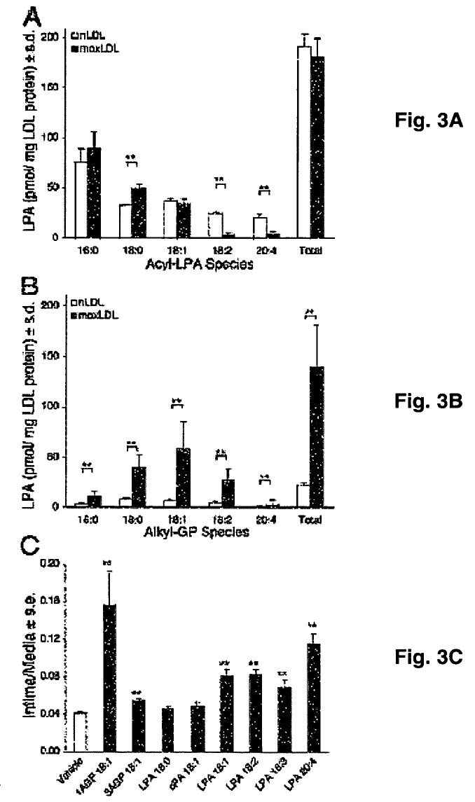

The concentrations of five acyl-lysophosphatidic acid (acyl-LPA) species were determined in native LDL and minimally oxidized LDL after scopper-mediated minimal oxidization (FIG. 3A). Surprisingly, total acyl-LPA levels in minimally oxidized LDL were not significantly different (180±19 pmol/mg LDL protein, n=4) from the native LDL control (190±13 pmol/mg LDL protein, n=4). However, there were significant decreases in the concentration of polyunsaturated acyl-LPA species in minimally oxidized LDL, a finding consistent with oxidative degradation.

Although the majority of lysophosphatidic acid characterized in biological fluids and tissues is in the acyl form, the alkyl ether glycerophosphate analogue, alkyl-GP, has also been detected. Alkyl-GP has biological properties distinct from acyl-LPA. For example, alkyl-GP is 50 times more potent than acyl-LPA in the activation of platelets. Alkyl-GP levels were quantified in the LDL preparations and it was found that alkyl-GP content was six-fold higher in minimally oxidized LDL, with the octadecenenyl (18:1) species showing a 10-fold increase over native LDL (FIG. 1E and FIG. 3B). Interestingly, the rank order of alkyl-GP species present in minimally oxidized LDL was the same as reported for the lipid core of human atherosclerotic plaques.

### EXAMPLE 5

The Role of Lysophosphatidic Acid in Neointima Formation in Rat

Mild oxidation of LDL produces prothrombotic and proatherogenic minimally oxidized LDL. Lysophosphatidic acid G protein-coupled receptor antagonist abolishes platelet aggregation elicited by minimally oxidized LDL, indicating that lysophosphatidic acid plays an essential role in the thrombogenic effects of minimally oxidized LDL. To define the contribution of lysophosphatidic acid to the neointima-inducing potential of minimally oxidized LDL, the effects of various alkyl ether glycerophosphate (alkyl-GP) and acyl-LPA species on neointima formation in the rat carotid artery model were determined (FIG. 3C). 1-O-octadecenyl-glycerophosphate (1AGP; the natural stereoisomer) was highly effective, whereas 3-O-octadecylglycerophosphate (3AGP; the unnatural stereoisomer) was modestly effective in eliciting neointima. The ether bond in alkyl-GP is resistant to cleavage by phospholipases A. Consequently, the metabolic conversion of alkyl-GP-derived fatty alcohols can be ruled out, suggesting that intracellular phospholipases of the A type are not involved in generating a bioactive metabolite of alkyl-GP. 2,3-cyclic phosphatidic acid (cPA; 18:1), an endogenous, unsaturated acyl-LPA analogue containing a cyclic phosphate, was inactive. Unlike alkyl-GP, cPA18:1 is a substrate for phospholipases A.

Hence, the lack of its neointima-inducing action indicates that oleic acid, a potential hydrolysis product of cPA 18:1, is not sufficient to elicit this response. Together, these results indicate a stereospecific requirement for both an unsaturated fatty acid/fatty alcohol group and a free phosphate on the glycerol backbone to stimulate neointima formation.

Activation of platelets results in lysophosphatidic acid production, which is dominated by the polyunsaturated 20:4 (arachidonoyl) and 18:2 (linolenoyl) acyl species. It has been shown previously that lysophosphatidic acid species containing unsaturated fatty acyl groups 16:1, 18:1, and 18:2 induced formation of neointimal lesions, whereas the saturated acyl-LPA species were inactive (Yoshida et al., 2003).

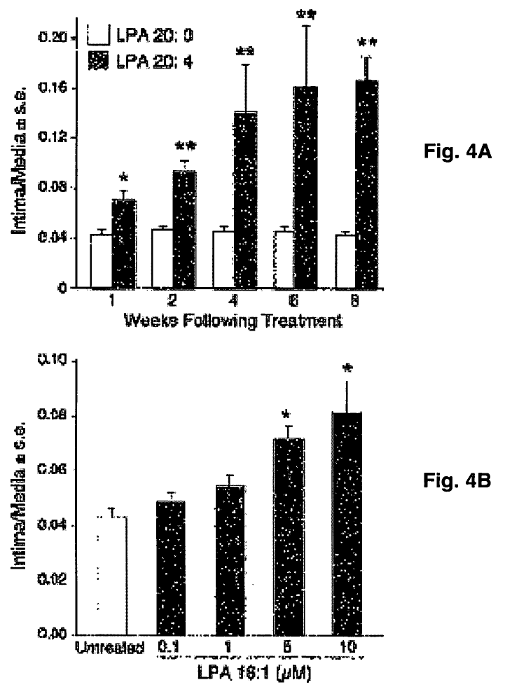

Rat carotid arteries were exposed for 1 hour to lysophosphatidic acid 20:4 or 20:0 and vascular remodeling was monitored for up to 8 wk thereafter. Lysophosphatidic 20:4 was chosen because it is the most abundant species in human serum (˜40% of total) with concentrations up to 2.5 μM. In contrast, the total circulating concentration of acyl-LPA in plasma is <0.1 μM. This brief exposure to 2.5 μM lysophosphatidic acid 20:4 elicited progressive neointimal growth, whereas 2.5 μM lysophosphatidic acid 20:0 was completely inactive (FIG. 4A). The extent of neointima development elicited by lysophosphatidic acid 18:1 treatment was concentration dependent up to 10 μM, the highest concentration tested and reached statistical significance at 5 μM (P<0.01; FIG. 4B). This concentration is equivalent to the total LPA concentration found in human serum.

### EXAMPLE 6

The Role of G Protein-Coupled Receptors In Lysophosphatidic Acid-Induced Neointima Formation

Expression of lysophosphatidic acid-specific G protein-coupled plasma membrane receptors in rat carotid arteries was examined by RT-PCR as described earlier by Wang et al. (2002). To quantify heavy caldesmon mRNA, quantitative PCR was performed applying the real-time SYBR Green PCR method using a Sequence Detection System Model 7700 (Applied Biosystems) instrument. Rat heavy caldesmon and GAPDH (reference control mRNA)-specific primers were designed with Primer Express Software (Applied Biosystems), and forward and reverse primers were as follows: 5′-GAACCAAAGCTGAGCAGGACA-3′ (SEQ ID NO:1) and 5′-TTCGTGCAGCCTCCATTCTT-3′ (SEQ ID NO:2) for heavy caldesmon; 5′-AAGCTCACTGGCATGGCCTT-3′ (SEQ ID NO:3) and 5′-CGGCATGTCAGATCCACAAC-3′ (SEQ ID NO:4) for GAPDH. Amplification reaction was performed with SYBR Green PCR Master Mix (Applied Biosystems) following the manufacturer's protocol. mRNA abundance calculation was based on Ct values as described previously (Wang et al., 2002). The expression level of heavy caldesmon mRNA was normalized to GAPDH mRNA. Each PCR reaction was performed at least three times, and the result was expressed as mean±SEM. Statistical comparison of mRNA expression was evaluated by ANOVA, and P<0.05 was considered statistically significant.

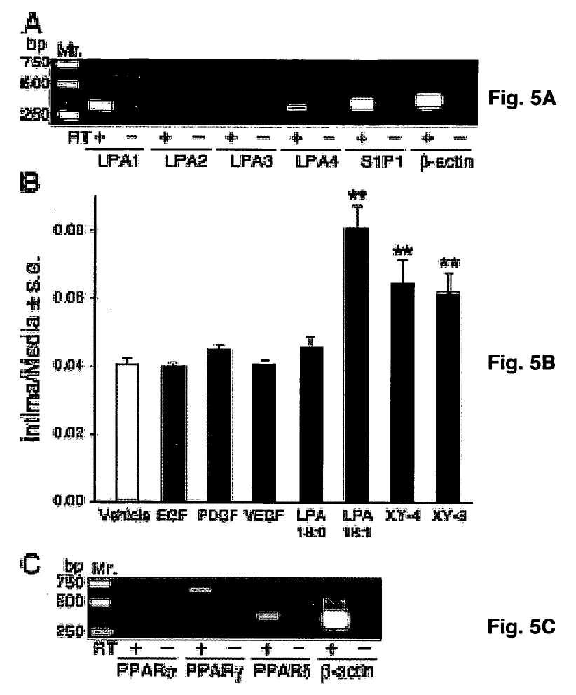

Lysophosphatidic acid is a growth factor-like phospholipid mediator that activates specific G protein-coupled plasma membrane receptors LPA₁, LPA₂, and LPA₃ encoded by the endothelial differentiation gene family and the distantly related LPA₄. RT-PCR analysis using gene-specific primers showed dominant expression of LPA₁, low levels of LPA₄, and no transcripts for LPA₂ or LPA₃ in untreated rat carotid arteries (FIGS. 3A and 5A).

Lysophosphatidic acid-specific G protein-coupled plasma membrane receptors are activated by both saturated and unsaturated acyl-LPA species with a rank order of potency of acyl-LPA>alkyl-GP>cPA. Moreover, lysophosphatidic acid-specific G protein-coupled receptors do not show stereoselectivity to alkyl-GP. The structure-activity relationship for lysophosphatidic acid-induced neointima formation was markedly different from that described for the G protein-coupled receptors. First, neointima formation shows a rank order of alkyl-GP>acyl-LPA, with cPA being inactive. Second, unsaturated but not saturated fatty acyl species stimulated lesion formation (FIG. 3C). Third, formation of neointima shows a stereoselective preference for 1-O-octadecenyl-GP over 3-O-octadecenyl-GP. Fourth, in contrast to alkyl-GP and unsaturated acyl-LPA, EGF (50 ng/ml), VEGF (10 ng/ml), and PDGF (10 ng/ml) failed to induce detectable neointima in this model after a 2-wk period (FIG. 5B). Lysophosphatidic acid G protein-coupled receptors have been found to transactivate the EGF and PDGF receptors; thus, the lack of neointimal response to authentic ligands of these tyrosine kinase receptors discredits the involvement of such a mechanism. Fifth, fluorinated lysophosphatidic acid-like PPARγ agonists that are four orders of magnitude less potent as lysophosphatidic acid G protein-coupled receptor agonists than lysophosphatidic acid 18:1 were nearly as potent at inducing neointima formation in the rat model (FIG. 5B). These results lead to the conclusion that a receptor distinct from known lysophosphatidic acid G protein-coupled receptors mediates neointimal lesion formation.

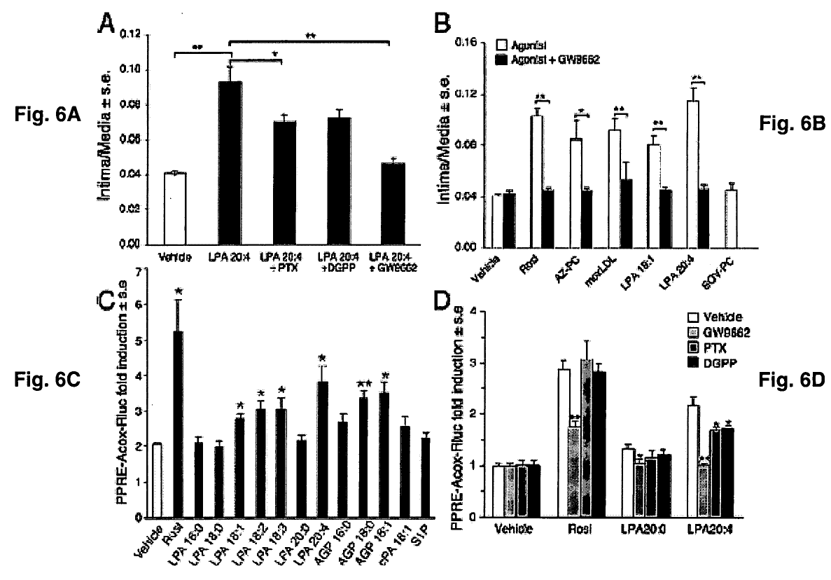

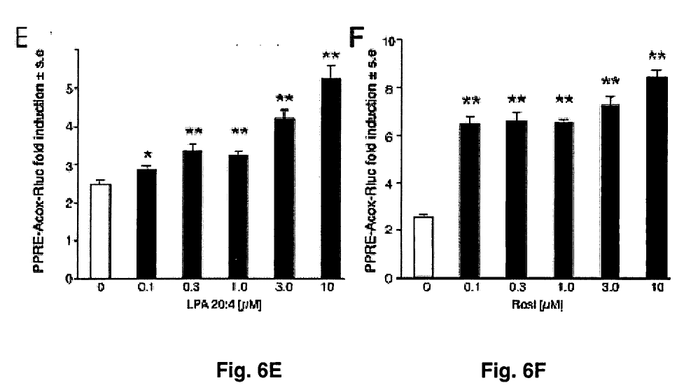

G protein-coupled receptor LPA₁-induced cell proliferation is fully blocked by pertussis toxin (PTX). To examine the role of pertussis toxin-sensitive G proteins in the neointimal response elicited by lysophosphatidic acid, vessels were treated with 100 ng/ml pertussis toxin for 30 min before and during lysophosphatidic acid 20:4 exposure. Pertussis toxin pretreatment attenuated, but did not abolish, the response to lysophosphatidic acid 20:4 (FIGS. 3A and 6A). Among lysophosphatidic acid G protein-coupled receptors, the LPA₃ receptor is unique with a preference for unsaturated lysophosphatidic acid species. However, RT-PCR analysis of normal carotid tissue showed no detectable LPA₃ transcript (FIG. 5A). Likewise, dioctylglycerol pyrophosphate (DGPP), a competitive antagonist of the LPA₃ and LPA₁ receptors, produced a modest inhibition (FIG. 6A). These observations discount, but do not completely exclude, a major role for lysophosphatidic acid G protein-coupled receptors in neointima formation.

### EXAMPLE 7

The Role of Peroxisome Proliferator Activated Receptor-γ in Lysophosphatidic Acid-Induced Neointima Formation

In addition to its plasma membrane receptors, lysophosphatidic acid was shown recently to be an agonist of the nuclear transcription factor peroxisome proliferator activated receptor-γ (PPARγ), which has long been implicated in atherogenesis. Many compounds activate peroxisome proliferator activated receptors, including the synthetic drug Rosi of the thiazolidinedione (TZD) family, oxidized phospholipids, fatty acids, eicosanoids, and oxidized LDL. PPARγ has been detected in macrophages/monocytes, vascular smooth muscle cells, endothelial cells, and is highly expressed in atherosclerotic lesions and hypertensive vascular wall. PPARγ was detected in normal rat carotid tissue by RT-PCR (FIG. 5C). For this reason, GW9662, a specific irreversible antagonist of PPARγ, was applied at a concentration of 5 μM 30 min before and was coapplied with 2.5 μM lysophosphatidic acid 20:4. GW9662 completely abolished neointima formation elicited by lysophosphatidic acid 20:4, indicating that PPARγ activation is required for the development of lysophosphatidic acid-induced lesion development (FIG. 6B).

To further evaluate the hypothesis that PPARγ activation leads to neointima formation, vessels were pretreated with GW9662 or vehicle control, with subsequent coapplication of the PPARγ agonist Rosi. Rosi induced neointima formation with a time course identical to that of LPA 20:4, indicating that the activation of PPARγ is sufficient to elicit neointima formation in this model (FIG. 6B). Lesion formation by Rosi was fully blocked by GW9662 treatment (FIG. 6B). Endogenous PPARγ agonist 1-O-hexadecyl-2-azale-oly-phosphatidylcholine (AZ-PC), an active component of minimally oxidized LDL, also caused neointima formation that was inhibited by GW9662 (FIG. 6B). Accordingly, neointima formation in response to minimally oxidized LDL, LPA 20:4, and LPA 18:2 was also inhibited by GW9662 pretreatment. In contrast, treatment with the PPARγ agonist stearoyl-oxovaleryl phosphatidylcholine (SOV-PC) and saturated lysophosphatidic acid species failed to stimulate neointima formation, and GW9662 treatment had no effect on vessels from animals treated with these agents (FIG. 6B). These results together with those obtained with the PPARγ agonists XY4 and XY8 (FIG. 5B) support an essential role for PPARγ activation in neointima formation in the rat model.

The structural requirements of lysophosphatidic acid-elicited neointima formation are distinct from those of any known lysophosphatidic acid G protein-coupled receptors. Therefore, the investigators compared the unique in vivo neointima-eliciting lysophosphatidic acid structure-activity relationship with that of PPARγ using an in vitro assay that utilizes an acyl-coenzyme A oxidase-luciferase (PPRE-Acox-Rluc) reporter gene construct containing a peroxisome proliferator-activated receptor response element. CV-1 cells were plated in 96-well plates (5×10³ cells per well) in DME supplemented with 10% FBS. The next day, the cells were transiently transfected with 125 ng of pGL3-PPRE-acyl-CoA oxidase (Acox)-renilla luciferase), or 125 ng pGL3-CD36(−273), or pGL3-CD36(−261), 62.5 ng of pcDNAI-PPARγ, and 12.5 ng of pSV-β-galactosidase (Promega) using LipofectAMINE™ 2000 (Invitrogen). Twenty four hours after transfection, cells were treated with 1% FBS-supplemented OptiMEMI™ (Invitrogen) containing DMSO or test compound (10 μM) in DMSO for 20 h. Luciferase and β-galactosidase activities were measured with the Steady-Glo® Luciferase Assay System (Promega) and the Galacto-Light Plus™ System (Applied Biosystems), respectively. Samples were run in quadruplicate, and the mean±SE were calculated. Data are representative of at least three independent transfections. Student's t test was used for null hypothesis testing, and P<0.05 was considered significant.

Results from this assay (FIG. 6C) were identical to the structure-activity relationship found in vivo (FIG. 3C). Rosi, unsaturated acyl-LPA species, and alkyl-GP all elicited significant activation of the PPRE-Acox-Rluc reporter, whereas saturated acyl LPA species, cPA, and the related lipid mediator S1P were inactive. Interestingly, not only octadecenyl-GP but also octadecyl-GP and hexadecyl-GP activated the reporter gene, indicating the unique properties of alkyl-GP analogs in activating PPARγ. Moreover, pertussis toxin and DGPP treatment reduced but did not abolish activation of the PPRE-Acox-Rluc reporter (FIG. 6D), results that are consistent with the in vivo experiments (FIG. 6A). The dose-response relationship of lysophosphatidic acid- and Rosi-elicited activation of the PPRE-Acox-Rluc reporter (FIGS. 6E and F) was similar to that of the neointimal response (FIG. 4B); although with a lower threshold, as high nanomolar concentrations of the two PPARγ agonists were sufficient to cause a significant activation of the receptor genes.

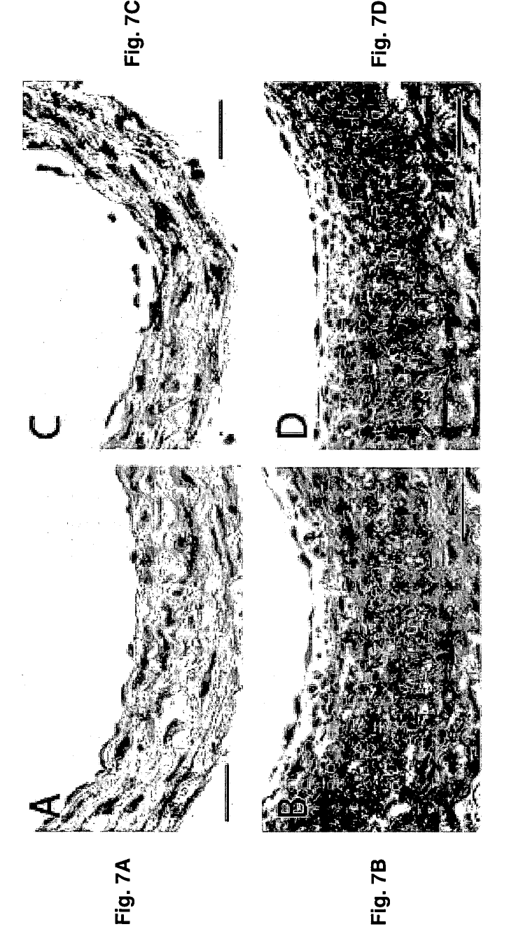

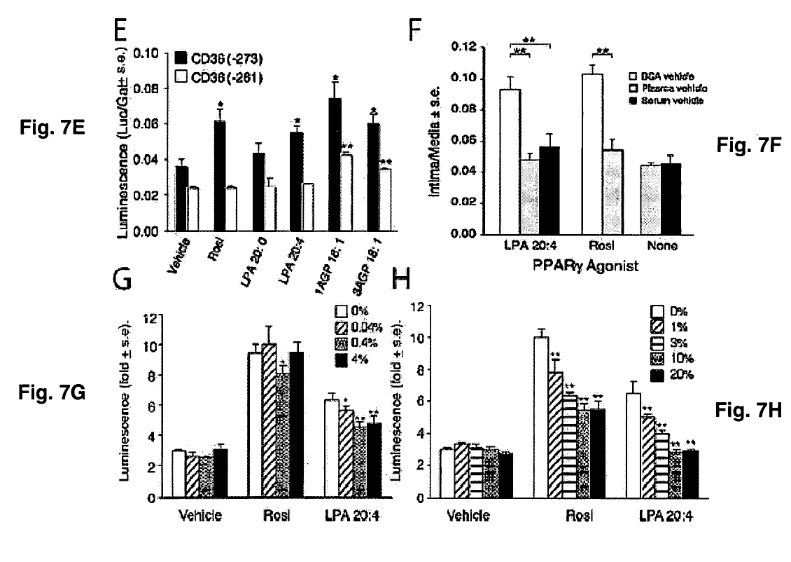

Rosi, LPA 20:4, and alkyl-GP, but not LPA 20:0, up-regulated the expression of a CD36-luc reporter gene when tested in vitro using CV1 cells transfected with one of two CD36-luc reporter constructs, one with and one without the peroxisome proliferator-activated receptor (PPAR) response element (PPRE) between −273 and −261 (FIG. 7E). This activation was dependent on the presence of the PPRE because neither Rosi nor lysophosphatidic acid 20:4 activated the reporter gene with a deleted PPRE. Interestingly, whereas activation by alkyl-GP was much reduced in the PP deletion mutant it was not completely abolished, suggesting that in CV-1 cells other promoter elements might also become activated by this ligand but not by lysophosphatidic acid 20:4.

### EXAMPLE 8

Expression of Peroxisome Proliferator Activated Receptor-γ in Rat Carotid Arteries

Expression of PPARγ protein was examined in rat carotid arteries exposed to lysophosphatidic acid or minimally oxidized LDL. Immunohistological staining for the PPARγ antigen showed only a few stained nuclei in arteries from animals treated with LPA 20:0 (FIG. 7A) or native LDL (data not shown). In contrast, intense PPARγ immunoreactivity was observed in neointimal lesions carotid arteries from the LPA 20:4 (FIG. 7B) or minimally oxidized LDL (data not shown) treatment groups that strongly resembled that reported previously in atherosclerotic lesions.

The scavenger receptor CD36, a PPARγ-regulated gene, contains a PPAR response element within its promoter between base pairs −273 and −261. CD36 plays a critical role in lipid uptake by binding and transporting oxidized lipids, including minimally oxidized LDL. Thus, this pathway could provide a source of ligands, such as lysophosphatidic acid, alkyl-GP, and AZ-PC, to sustain PPARγ activation. Acyl and alkyl forms of lysophosphatidic acid both accumulate in human atherosclerotic plaques. Lysophosphatidic acid activates CD36-mediated lipid uptake into macrophages through a PPARγ-PPRE-dependent mechanism. Immunostaining for the CD36 antigen showed no increase in immunoreactivity in vessels treated with lysophosphatidic acid 20:0 (FIG. 3F and FIG. 7C) or native LDL (data not shown). However, neointimal tissue derived from treatment with lysophosphatidic acid 20:4 (FIG. 7D) or minimally oxidized LDL (data not shown) showed intense CD36 immunoreactivity. The up-regulated expression of CD36 suggests that PPARγ is activated in LPA 20:4- and minimally oxidized LDL-elicited neointimal lesions.

### EXAMPLE 9

Effects of Serum Factor on Lysophosphatidic Acid-Induced Neointima Formation

There is 0.1 μM LPA in plasma and thiazolidinedione (TZD) is now being used for the treatment of diabetics, yet there are no reports of increased vascular complications in patients. To resolve this apparent controversy, the investigators tested whether plasma factors could attenuate the neointima-inducing effects of Rosi and/or lysophosphatidic acid. This hypothesis is based on earlier reports showing that lysophosphatidic acid diluted in plasma and high concentrations of albumin shows diminished biological responses. Heparinized, syngeneic plasma, or serum was compared with serum albumin (10 μM) as the vehicle for Rosi and lysophosphatidic acid 20:4 delivery. Only those animals receiving Rosi or lysophosphatidic acid 20:4 complexed with 10 μM albumin developed neointimal lesions, whereas those that received the compounds delivered in plasma or serum showed no significant neointima formation (FIG. 7F). Neither plasma, nor serum alone had neointima-inducing effects (FIG. 7F). These results suggest that plasma and serum factors attenuate formation of neointima and serve to mitigate widespread effects of endogenous lysophosphatidic acid and suppress the effect of Rosi. LPA presents in serum readily activates LPA G protein coupled receptor (GPCR)-mediated biological responses; thus, the lack of activity of serum and LPA delivered in serum points to an important difference in ligand recognition of lysophosphatidic acid GPCR versus PPARγ. Moreover, it has been reported that transbilayer movement of alkyl-GP is blocked by high concentrations (2%) of albumin, whereas substantial movement remains at low albumin concentrations (0.05%), which is similar to what was used in the assays described above.

To further substantiate the notion that transbilayer movement of lysophosphatidic acid and Rosi could be affected by albumin and plasma/serum factors in a concentration-dependent manner, the PPRE-Acox-Rluc reporter assay was used. Addition of albumin (0.04-4% wt/vol; FIG. 7G) or rat serum (1-20% vol/vol; FIG. 7 H) inhibited lysophosphatidic acid and Rosi-induced activation of the PPRE-Acox-Rluc reporter gene in a concentration-dependent manner, providing further support to the hypothesis that carrier proteins could provide a physiological barrier to the transbilayer movement of these ligands, thus preventing/attenuating activation of PPARγ.

### EXAMPLE 10

Phenotypic Modulation of Vascular Smooth Muscle Cells by Peroxisome Proliferator Activated Receptor-γ Agonists

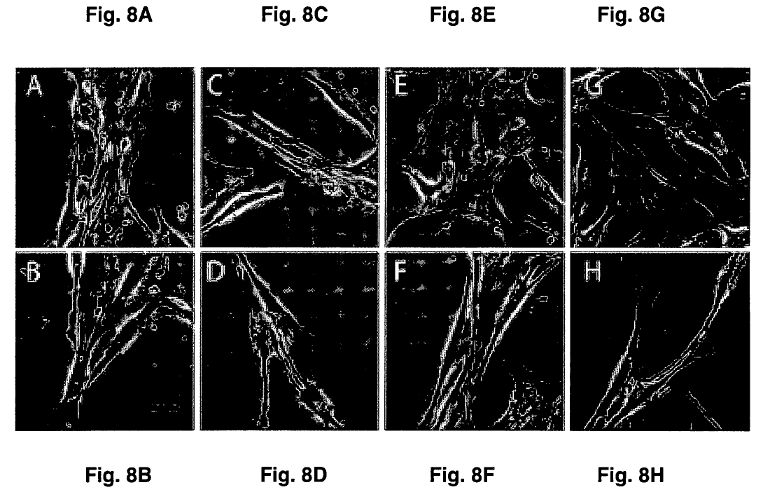

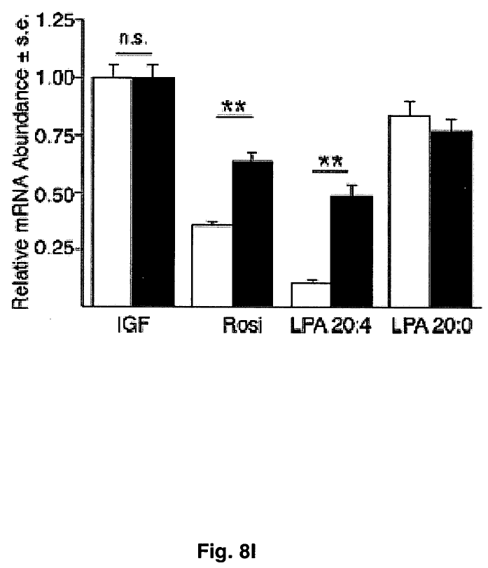

It has been reported that unsaturated lysophosphatidic acid species induced phenotypic modulation of cultured vascular smooth muscle cells (VSMCs). Because the neointimal response and PPARγ display a similar selectivity for unsaturated lysophosphatidic acid, that activation of PPARγ plays a role in the phenotypic dedifferentiation is hypothesized herein. This hypothesis was tested in a culture system in which IGF-1 maintains the differentiated spindle shape of vascular smooth muscle cells as indicated by high levels of heavy caldesmon (hCAD) mRNA expression. Vascular smooth muscle cells established in the presence of 2 ng/ml IGF-1 for 2 days were exposed to 1 μM of either Rosi, LPA 20:4, or LPA 20:0 for 3 days with or without 200 nM GW9662. Those vascular smooth muscle cells exposed to Rosi and LPA 20:4 developed a fibroblast-like flattened morphology (FIGS. 8E and G), whereas cultures exposed to lysophosphatidic acid 20:0 (FIG. 8C) maintained the spindle-like differentiated morphology seen in the IGF-1-treated controls (FIG. 8A). GW9662 treatment reversed this effect of LPA 20:4 and Rosi (FIGS. 8F and H). Using quantitative RT-PCR, it was found that hCAD mRNA expression decreased by all treatments but was most pronounced in vascular smooth muscle cells exposed to LPA 20:4 and Rosi (FIG. 8I). GW treatment caused a significant increase in hCAD mRNA expression. These results support the hypothesis that PPARγ plays an essential role in the phenotypic modulation of vascular smooth muscle cells.

### EXAMPLE 11

Screening of Lysophosphatidic Acid Analogs, Peroxisome Proliferator Activated Receptor-γ Agonists and Antagonists

Three assays can be used: 1) Competition binding to purified recombinant PPARγ against [³²P]-LPA as described previously (McIntyre et al., 2003); 2) PPARγ-mediated activation of an acyl-CoA oxidase-luciferase reporter gene with peroxisomal proliferators-responsive elements (McIntyre et al., 2003); and 3) Neointima induction in rats as described above.

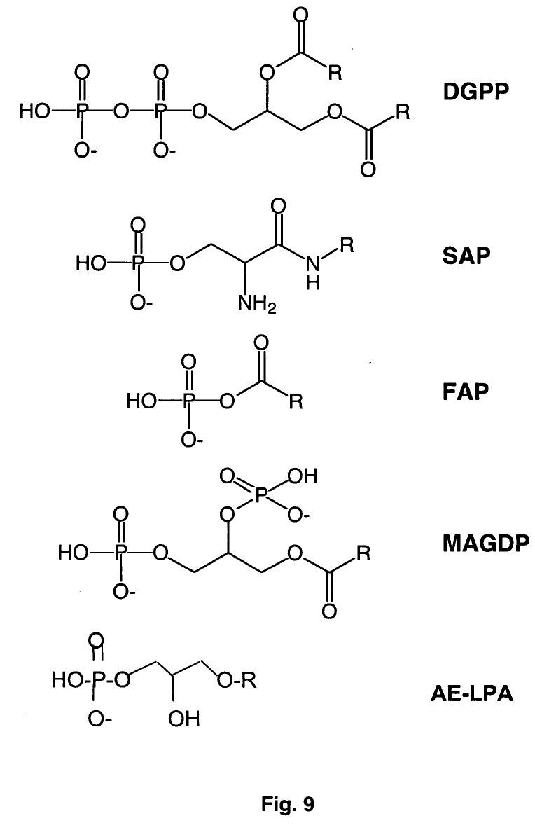

Four lead structures (FIG. 9) that were identified earlier either as agonists or antagonists of lysophosphatidic acid G protein-coupled receptor will be examined. These are: 1) diacylglycerol pyrophosphate (DGPP) (Fischer et al., 2001); 2) serine-phosphoric acids (SAP) (Bittman et al., 1996; Liliom et al., 1996); 3) fatty alcohol phosphates (FAP) (Virag et al., 2003) and 4) monoacylglycerol-diphosphates (MAGDP).

Short chain diacylglycerol pyrophosphates are competitive inhibitors with a 10-fold preference for LPA₃ over LPA₁ but without effect on LPA₂. Serine-phosphoric acids inhibit all three lysophosphatidic acid receptors, whereas fatty alcohol phosphate 12:0 is an antagonist for LPA₃ and a specific agonist for LPA₂ without any effect on LPA₁. Monoacylglycerol-diphosphates analogs inhibit LPA₁. The inventors have generated over a hundred analogs of these lead structures, many with unsaturated aliphatic chains that are potential ligands of PPARγ.

Competition binding between [³²P]-LPA and different concentrations of unlabeled analogs can be measured by binding to His₆-PPARγ immobilized on Ni-Sepharose beads (McIntyre et al., 2003). The PPARγ-specific ligand Rosiglitazone will serve as positive control that competes against lysophosphatidic acid. Functional activity of the ligands on the PPARγ-PPRE system can be assayed in RH7777 cells that carry no lysophosphatidic acid plasma membrane receptors. RH7777 cells can be transiently transfected with 0.1 μg of simian virus 40-β-galactosidase reporter and 1 μg of acyl-CoA oxidase-luciferase. The acyl-CoA oxidase-luciferase construct has a peroxisomal proliferator-responsive element (PPRE) and can be transactivated by the PPARγ/RXR transcription heterodimer. The ratio of β-galactosidase activity (a measure of transfection efficiency) and the luciferase luminescence (determined by transcriptional upregulation) is a normalized measure of PPARγ activation. The competition assay can be carried out on high-throughput screening equipment such as Brandel 96-well harvester with automatic filter processor (Fusion Alpha Plate Fluorescence/luminescence reader, Packard Inc.). There are more LPA analogs and other leads including the sn2-difluoromethyl-lysophosphatidic acid analogs that activate PPARγ without activating LPA G protein-coupled receptor. Those analogs that are found to activate PPARγ will also be assayed in vivo for neointima induction in rats to provide a correlative measure between in vitro and in vivo activity.

Results from these experiments will determine whether a given analog: 1) binds to PPARγ; 2) is an agonist of PPARγ; and 3) whether in vitro PPARγ-ligand activity of a given compound correlates with its in vivo activity. If a compound binds to PPARγ but is inactive in the PPARγ/PPRE activation assay, suggesting that it is a potential antagonist, it will be further tested in the activation assays against Rosiglitazone. RH7777 cells that do not express LPA G protein-coupled receptor but express PPARα, PPARδ, and PPARγ are ideal for these assays.

The following references were cited herein:

Davies et al., Oxidized alkyl phospholipids are specific, high affinity peroxisome proliferator-activated receptor gamma ligands and agonists. J. Biol. Chem. 276:16015-16023 (2001).

Bittman et al., Inhibitors of lipid phosphatidate receptors: N-palmitoyl-serine and N-palmitoyl-tyrosine phosphoric acids. J Lipid Res. 37:391-398 (1996).

Fischer et al., Mol. Pharmacol. 60:776-784 (2001).

Liliom et al., Mol. Pharmacol. 50:616-623 (1996).

McIntyre et al., Proc. Natl. Acad. Sci. USA. 100:131-136 (2003).

Virag et al., Mol. Pharm. (2003).

Wang et al., Am. J. Physiol. Cell Physiol. 283:C1646-C1654 (2002).

Xu and Prestwich, Org. Lett. 4:4021-4024 (2002).

Yokoyama et al., Biochim. Biophys. Acta. 1582:295-308 (2002).

Yoshida et al., Circulation 108:1746-1752 (2003).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of inhibiting neointima formation in a subject, said method comprises the step of administering to said subject a compound that inhibits signaling through peroxisome proliferator-activated receptor gamma (PPARγ).

2. The method of claim 1, wherein said compound is a peroxisome proliferator-activated receptor gamma antagonist.

3. The method of claim 1, wherein said compound is GW9662.

4. The method of claim 3, wherein said GW9662 is administered in a dose of from about 0.01 mg/kg to about 500 mg/kg of the subject's body weight.

5. The method of claim 1, wherein said compound is a analog of lysophosphatidic acid (LPA), said analog binding to but not activating PPARγ.

6. The method of claim 5, wherein said LPA analog comprises one or two unsaturated carbon chains or one or two saturated carbon chains or a combination thereof.

7. The method of claim 6, wherein said analog is selected from the group consisting of diacylglycerol pyrophosphate, serine-phosphoric acids, fatty alcohol phosphates, alkyl ether glycerophosphates, and monoacylglycerol-diphosphates.

8. The method of claim 5, wherein said analog is administered in a dose of from about 0.01 mg/kg to about 500 mg/kg of the subject's body weight.

9. The method of claim 1, wherein said subject is an animal or a human.

10. A method of preventing or treating atherosclerosis in a subject, said method comprises the step of administering to said subject a compound that inhibits signaling through peroxisome proliferator-activated receptor gamma (PPARγ).

11. The method of claim 10, wherein said compound is a peroxisome proliferator-activated receptor gamma antagonist.

12. The method of claim 10, wherein said compound is GW9662.

13. The method of claim 12, wherein said GW9662 is administered in a dose of from about 0.01 mg/kg to about 500 mg/kg of the subject's body weight.

14. The method of claim 10, wherein said compound is an analog of lysophosphatidic acid (LPA), said LPA analog binding to but not activating PPARγ.

15. The method of claim 14, wherein said LPA analog comprises one or two unsaturated carbon chains or one or two saturated carbon chains or a combination thereof.

16. The method of claim 15, wherein said analog is selected from the group consisting of diacylglycerol pyrophosphate, serine-phosphoric acids, fatty alcohol phosphates, alkyl ether glycerophosphates, and monoacylglycerol-diphosphates.

17. The method of claim 14, wherein said analog is administered in a dose of from about 0.01 mg/kg to about 500 mg/kg of the subject's body weight.

18. The method of claim 10, wherein said subject is an animal or a human.
